(19) United States
(12) Patent Application Publication
(10) Pub. No.: US 2005/0142111 A1
(43) Pub. Date: Jun. 30, 2005

(54) Method to prevent transplant rejection by stable expression of heme oxygenase-1

(75) Inventors: Xiaobing Wu, Beijing (CN); Tung-Yu Tsui, Hong Kong (CN)

Correspondence Address: DARBY & DARBY P.C., P. O. BOX 5257, NEW YORK, NY, 10150-5257, US

(73) Assignee: AGTC GENE TECHNOLOGY COMPANY LTD., Beijing (CN)

(21) Appl. No.: 10/750,620

(22) Filed: Dec. 30, 2003

(51) Int. Cl. A61K 48/00; C12N 7/00; C12N 15/861

(52) U.S. Cl. 424/93.2; 435/456; 435/235.1

(57) ABSTRACT

The present invention relates to a method for the prevention of allograft rejection in clinical transplantation. Specifically, the method of the present invention relates to the prevention of transplant arteriosclerosis and interstitial fibrosis by stable and long-term expression of HO-1 in grafts. The present invention represents a novel therapeutic approach to prevent allograft rejection in clinical transplantation.

### FIELD OF THE INVENTION

The present invention relates to the field of biotechnology, and particularly to the preparation of a recombinant adeno-associated virus carrying heme oxygenase-1 (HO-1) gene (rAAV/HO-1). The present invention further relates to uses of the rAAV vector for mediation of the stable expression of HO-1 gene, and for the prevention of chronic graft rejection in organ transplantation. The present invention also indicates the essense of stable HO-1 expression and/or its enzymatic activity in both early and late survival of an organ transplant, and the prevention of transplant arteriosclerosis and interstitial fibrosis.

### BACKGROUND ART

Chronic allograft rejection is characterized by progressive impairment of graft function occurring months or years after organ transplant. This is a major issue affecting long-term therapeutic effect of organ transplant. Despite the introduction of new immunosuppressive agents and regimens, the incidence of chronic allograft rejection has not decreased significantly. Although the precise nature of molecular and cellular events of chronic allograft rejection is still unclear, it is generally believed that immune and non-immune factors cause the pathological processes such as vasculitis, graft injury, and the subsequent inflammatory reaction. The proliferation in the vessels of the graft, and the remodeling of graft tissue leading to interstitial fibrosis, substantively account for progressive graft dysfunction.

Previous studies have shown that the expression of the HO-1 gene increases in well preserved grafts, indicating its possible critical role in the protection of grafts. HO, as an important oxidative enzyme, controls heme catabolism. At present, three kinds of HO isozymes have been found. HO-1 differs from the other two HO isozymes in that it is inducible and can be expressed in various types of cells.

A wide range of stress-related stimuli can induce the expression of HO-1. HO-1 has potent cytoprotective effects that are likely mediated by its products including carbon monoxide, biliverdin/bilirubin and iron ions. The most extensively studied of these products, carbon monoxide, has significant anti-inflammatory activities. In animal studies, induction of HO-1 in grafts can protect them from ischemia/reperfusion injury and from acute graft rejection. Induction of HO-1 in the early days after transplantation can also ameliorate the development of antibody-associated chronic arteriosclerosis in an animal model of chronic allograft rejection using anti-CD4 monoclonal antibody. It is unclear whether expression of HO-1 is capable of promoting early graft survival and preventing chronic allograft rejection. In addition, the potential for HO-1 to induce long-term survival of an allogeneic organ graft has not been established. That is, the exact effect of long-term expression of HO-1 was unknown before the study of the present invention.

In this study, allografts were manipulated by HO-1 gene expression using a recombinant adeno-associated viral vector. This approach provided evidence as to when HO-1 expression is needed to suppress early graft rejection, when it is needed to suppress graft arteriosclerosis, and whether HO-1 by itself can lead to long-term survival of organ grafts. More importantly, the model adopted is one in which the only manipulation for the therapeutic group is induction of HO-1. Thus, this model evaluates the effects of the HO-1 gene by itself. Accordingly, this methodology may have potential for clinical transplantation.

Recombinant adeno-associated viral vectors (rAAV), as a vector for gene transfer and expression, is characterized by its capability of mediating long-term expression of the object gene, and introducing genes into both dividing and non-dividing cells. rAAV is the most attractive gene delivery system. This is because rAAV mediated transduction results in long-term expression and has no remarkable cytotoxicity. In the present study, we have demonstrated for the first time that an adeno-associated viral vector is highly effective in delivering protective genes to organ grafts for the prevention of acute and chronic allograft rejection. The inflammatory response, cellular damage, and tissue remodeling that lead to transplant arteriosclerosis and interstitial fibrosis, can be prevented by stable expression of HO-1 in the cells of grafts.

Previous studies have shown that over-expression of HO-1 protects grafts from ischemic/reperfusion injuries and from acute immune attacks. However, it is unclear when it is necessary to express HO-1 to achieve these beneficial effects. In addition, previous studies have shown that induction of the HO-1 gene prevents the development of arteriosclerosis. However, in the arteriosclerosis studies, HO-1 was induced only in the very first few days around transplant, and the combined administration of anti-CD4 antibodies or transfusion of donor's blood plus anti-CD40 ligand antibodies potentially obscured the effects of HO-1 itself. None of the previously performed studies shed light on when it is necessary to increase HO activity to prevent arteriosclerosis.

In the present studies, we attempt to use a clinically applicable model to investigate whether HO-1 expression alone can lead to long-term survival of grafts. Here, the results show that only 50-60% of long-term survival is achieved by HO-1 expression alone. Long-term survival increases to around 90% with an additional low dose of Cyclosporin A (CsA) administered for a total of 5 days.

In the present invention, production of rAAV/HO-1 is carried out by using a production system such as, for example, a recombinant adeno-associated virus vector and technology invented by WU, XiaoBing et al (China Patent Appl. No.99119039.4 and 99123723.4).

### SUMMARY OF THE INVENTION

Chronic allograft rejection is a major obstacle to organ transplantation for long-term treatment of end-stage organ failure. In this study, we transduced the anti-oxidant gene, heme oxygenase-1 (HO-1) to organ grafts using a recombinant adeno-associated viral vector (rAAV) in a rat transplantation model. The study demonstrates that long-term allograft survival can be achieved by rAAV mediated HO-1 gene expression. Furthermore, the long-term stable expression of HO-1 is a prerequisite for both early and late survival of the graft, and for prevention of transplant arteriosclerosis and interstitial fibrosis. rAAV/HO-1 mediated long-term graft protection is accompanied by down-regulation of genes encoding pro-inflammatory cytokines, growth factors, and tissue protease inhibitors; and by up-regulation of serine proteinases in the grafts. rAAV/HO-1 gene expression represents a novel therapeutic approach to prevent chronic allograft rejection in clinical transplantation.

### BRIEF DESCRIPTION OF THE DRAWINGS

FIG. 1a-FIG. 1h Show the Expression Profiles of rAAV Mediated Exogenous Gene:

FIG. 1a shows the gene expression frame of adeno-associated viral vector, wherein ITR, hCMV, GFP and rHO-1 respectively refer to the 145-basepair terminal repeats, human cytomegalovirus promoter, green fluorescent protein and rat heme oxygenase-1;

FIG. 1b shows the expression of GFP in the perivascular area of graft on day 7 after transplant. 1×10¹² vector genomes of rAAV/GFP were administered through the coronary artery of heart graft during cold preservation. Syngeneic heart transplantation was performed in LEW rats (original 400×);

FIG. 1C shows the expression of HO-1 detected in endothelial cells of the vessel of graft on day 7 after transplant (original 400×);

FIG. 1d & FIG. 1e respectively show the expression of GFP (FIG. 1d) and HO-1 (FIG. 1e) detected in the cardiomyocytes on day 30 after transplant (original 200×);

FIG. 1f shows content of HO-1 protein in the grafts (western blot analysis); the upper line represents the expression of HO-1 with molecular weight of 32 Kda; the lower one represents the expression of control β-protein (β-actin with molecular weight of 43 KDa); the first, the second, the third and the fourth line from the left respectively represent expressions on day 7, day 14, day 30 and day 100;

FIG. 1g shows the activity of HO-1 determined by measurement of carbon monoxide production (presented as carboxyhemoglobin in the peripheral blood of recipient animals), wherein the abscissa represents the lapse time after transplant (number of days) and the ordinate the content of carboxyhemoglobin (%). The level of carboxyhemoglobin increased in animals bearing rAAV/HO-1 treated grafts on day 14 (6.51±0.70%,n=5) and persisted in a stable level for 3 months;

FIG. 1h shows the intra-graft level of bilirubin which is significantly higher in rAAV/HO-1 treated grafts (1.32±0.09, n=5, P=0.0014) as compared with that in rAAV/GFP treated graft (0.94±0.05, n=5), wherein the left plot along the abscissa represents comparative rAAV/GFP treated grafts and the right one rAAV/GFP treated grafts, the ordinate represents level of bilirubin.

FIG. 2a-FIG. 2g Shows the Effects of rAAV/HO-1 Gene Expression in Allotransplantation Model:

FIG. 2a, wherein the abscissa represents the lapse time after transplant (number of days) and the ordinate the survival (%) of grafts. 57.1% of rAAV/HO-1 treated grafts survived over 100 days in the absence of any therapeutic intervention (MST>100 days, rAAV/HO-1, n=7, solid line), as compared with “None-treated” control group (MST: 21 days, n=7, long dashed line) and rAAV/GFP group (MST: 20 days, n=6, short dashed line), P<0.05;

FIG. 2b, wherein the abscissa represents the lapse time after transplant (number of days) and the ordinate the survival (%) of grafts. Combined use of rAAV/HO-1 treatment made the long-term survival of grafts receiving short-term of CsA (2.5 mg/kg, day 0-4) (MST>100 days, n=9, solid line) increasing from 50% of CsA alone group (MST>76.5, n=10, dashed line) to 88.9%, P<0.05;

FIG. 2c shows the serum level of anti-donor antibodies on day 100 detected by flow cytometric analysis, presented as ratio of values of sample/blank control (MCF, mean channel fluorescence); IgG1, IgG2b, IgM antibodies of rAAV/HO-1 treated group were significantly reduced as compared with those of rAAV/HO-1 group (n=5, P<0.05);

FIG. 2d & FIG. 2e show severity of intimal proliferation in the median arteries of grafts in CsA group (FIG. 2d) and CsA+rAAV/HO-1 group (FIG. 2e), displayed by Verhoeff's haematoxylin staining of elastin (black); these are representative pictures of continuous slices of 50 median arteries (5 grafts taken for each group);

FIG. 2f & FIG. 2g. show the immunofluorescence staining of α-smooth muscle actin (green) in the grafts of CsA (FIG. 2f) and CsA+rAAV/HO-1 (FIG. 2g) groups; these are representative pictures indicating the increase of deposition of α-smooth muscle actin in small arteries (FIG. 2j).

FIG. 3 Shows the Effect of Long-Term Expression of HO-1 Mediated by rAAV:

FIG. 3a-FIG. 3c show the effect of HO-1 inhibitor (ZnPPIX) to inhibit activity of HO-1 enzyme, detected by measuring the level of carboxyhemoglobin in the peripheral blood of recipients, wherein the levels of carboxyhemoglobin in CsA group and CsA+rAAV/HO-1 group are shown in FIG. 3a, and the levels of carboxyhemoglobin in CsA+rAAV/HO-1 group given ZnPPIX during day 1-30 and day 31-100 are respectively shown in FIG. 3b and FIG. 3c; data on each point is shown as mean±SD from 5 rats and the abscissa represents the lapse time after transplant (number of days) and the ordinate the level of carboxyhemoglobin (%);

FIG. 3d & FIG. 3e show the graft survivals of CsA group (n=6, dashed line) and CsA+rAAV/HO-1 group (n=6, solid line) with administration of ZnPPIX during day 0-30 (FIG. 3d) and day 31-100 (FIG. 3e) after transplant, wherein the abscissa represents the lapse time after transplant (number of days) and the ordinate the survival (%) of grafts.;

FIG. 3f & FIG. 3g show the comparison of severity of intimal proliferation in median arteries of grafts in rAAV/HO-1+CsA group after administration of ZnPPIX during day 0-30 (FIG. 3j) and day 31-100 (FIG. 3g) after transplant, after elastin staining by Verhoeff's haematoxylin;

FIG. 3h shows the grading of graft arteriosclerosis determined according to the severity of intimal proliferation in median arteries of grafts from different groups, wherein the first group from the left is for CsA group, the second one is for rAAV/HO-1+CsA group, the third one is for rAAV/HO-1+CsA group with administration of ZnPPIX during 1-30 days after transplant, and the fourth one is for rAAV/HO-1+CsA group with administration of ZnPPIX during day 30-100 after transplant, all the data is showed as Mean±S.E.M.
*P<0.001 versus CsA group,; **P=0.07 versus CsA group.

FIG. 4a-FIG. 4c Shows the Capability of HO-1 to Prevent the Development of Interstitial Fibrosis of the Grafts on day 100 after Transplant.

FIG. 4a shows Haematoxylin and eosin staining (original 100×) (upper row) and Masson's trichrome staining (original 200×) (lower row) of slices from grafts of CsA group (left column) and CsA+rAAV/HO-1 group (right column) on day 100 after transplant;

FIG. 4b shows the fibrotic areas (%) of slices from grafts of CsA group and CsA+rAAV/HO-1 group, the data are shown as Mean±SD from 20 slices/graft and 3 grafts/group, *P<0.001 versus CsA group;

FIG. 4c shows the immunofluorescence staining of collagen I (upper row) and fibronectin (lower row) in grafts of CsA group (left column) and rAAV/HO-1+CsA group (right column) on day 100 after transplant (FITC, original 200×).

FIG. 5a-FIG. 5.c shows the Under-Expression of mRNA of TGF-β1 and PAI-1 in the rAAV/HO-1 Treated Grafts on day 100 After Transplant:

FIG. 5.a & FIG. 5.b show mRNA expression levels of TGF-β1 (FIG. 5.a) and Smad protein family (FIG. 5.b) in the grafts on day 100 after transplant displayed in RNase protection assay, wherein the upper part of FIG. 5.a shows the expression level of TGF-β1 and the lower part that of control GAPDH, while from top to bottom of FIG. 5.b shows mRNA expression levels of Smad2, Smad3, Smad6, Smad7 and GAPDH.

FIG. 5.c shows the mRNA levels of PAI-1 (upper left 1), PAI-2 (upper left 2), tPA (upper right 1) and uPA (upper right 2) in grafts (5 for each group) detected by semi-quantitative RT-PCR, and the two lower ones show mRNA levels of control β-actin.

FIG. 6 shows the Structure of Plasmid pSNAV1/HO-1;

Table 1 is the gene expression profiling in rAAV/HO-1 treated grafts on day 100 after transplant. The data shown was selected from cDNA microarray data under the classification of growth factors, cytokines, chemokines, protease inhibitors and serine protease. The values determined were normalized with house-keeping genes and expression levels were considered as significant when the fold changes of gene transformation were greater or lower than 1 (log 2).

### DETAILED DESCRIPTION OF THE INVENTION

The present invention is published in Tung-YuTsui,MD; XiaobingWu,PhD; Chi-KeungLau,MPhil; David W. Y. Ho,BSc; Tao Xu,MB; Yeung-Tung Siu,MPhil; Sheung-Tat Fan,MD,PhD,FRCS, Circulation 2003; 107; 2623-2629.

In the present invention, the antioxidant gene HO-1 was inducted into organ grafts using a recombinant adeno-associated viral vector. Induction of the HO-1 gene achieves long-term expression of HO-1 in grafts, thereby demonstrating the effect of long-term expression of HO-1 on chronic allograft rejection. The results obtained show that expression of HO-1 mediated by an rAAV vector leads to long-term survival of grafts and prevention of graft arteriosclerosis and interstitial fibrosis. It has further been found that the long-term expression of HO-1 not only serves as a prerequisite for long-term survival of organ grafts, but also prevents transplant arteriosclerosis and interstitial fibrosis. rAAV/HO-1's long-term protection of grafts relates to under-expression of proinflammatory cytokines, growth factors, and tissue inhibitors of proteinases; and over-expression of serine proteases. The results obtained indicate that the rAAV mediated HO-1 gene expression during clinical organ transplant is a novel therapeutic approach for protection of chronic allograft rejection.

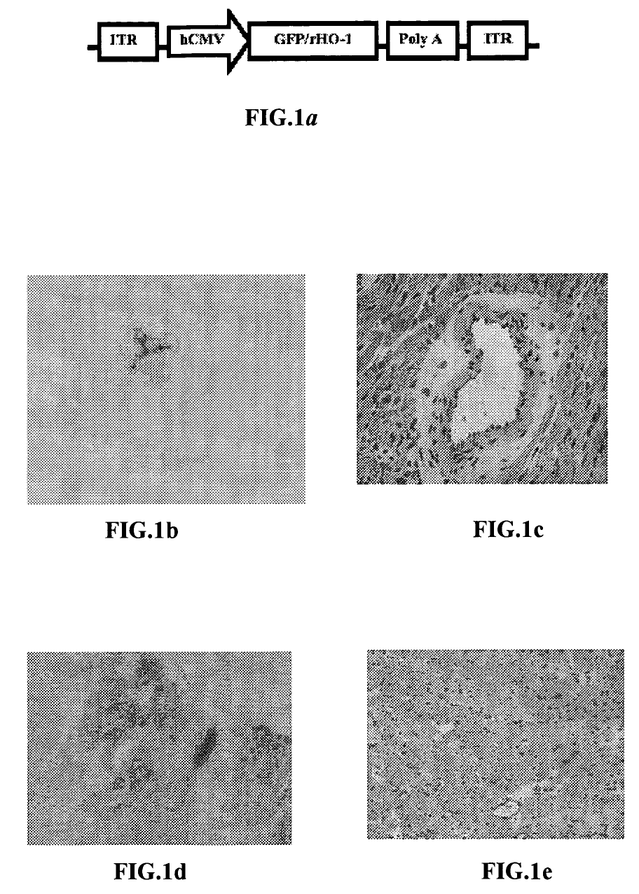

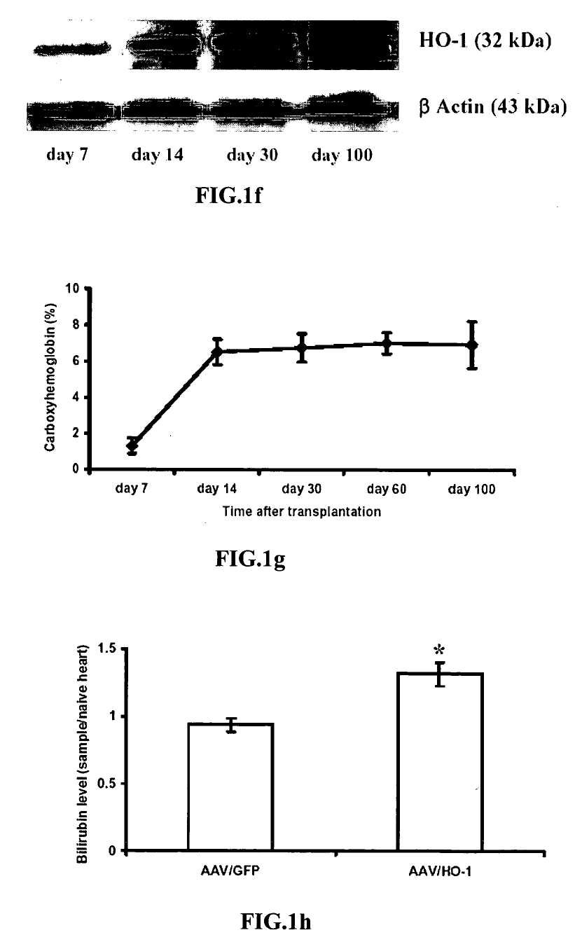

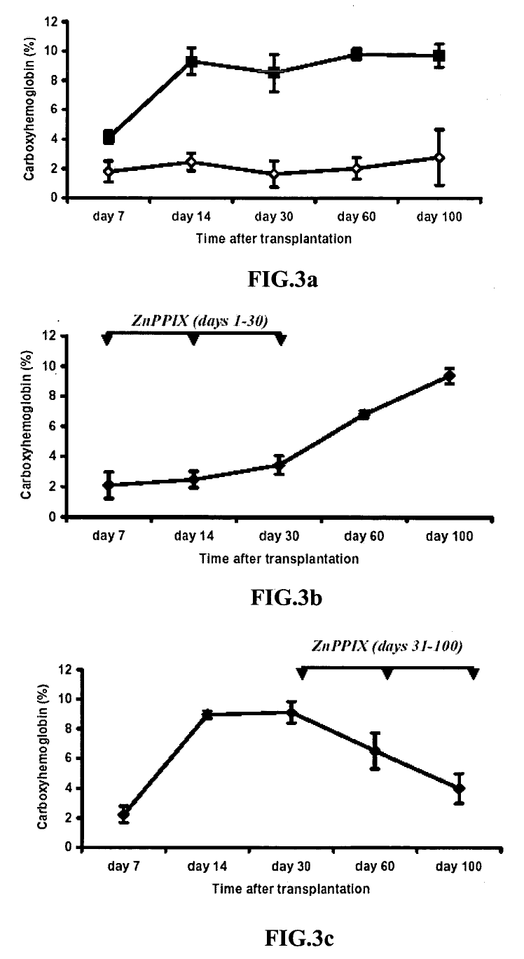

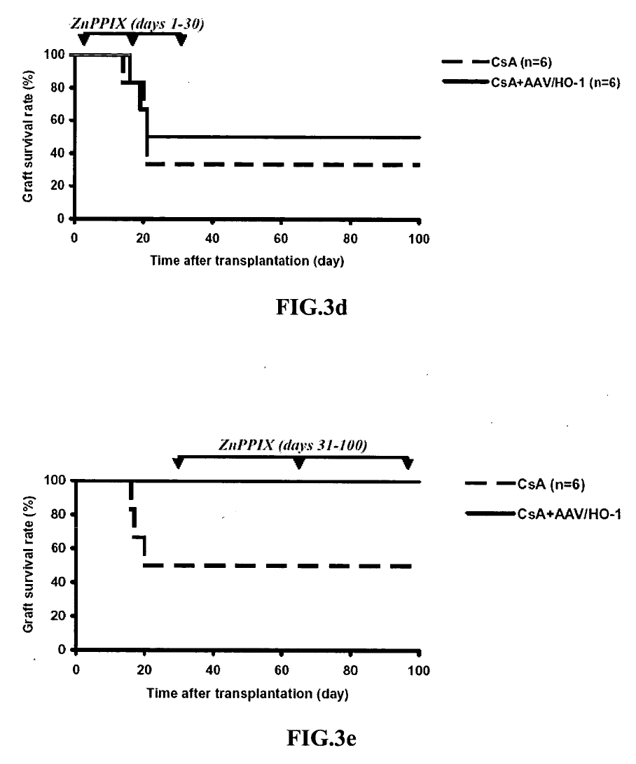

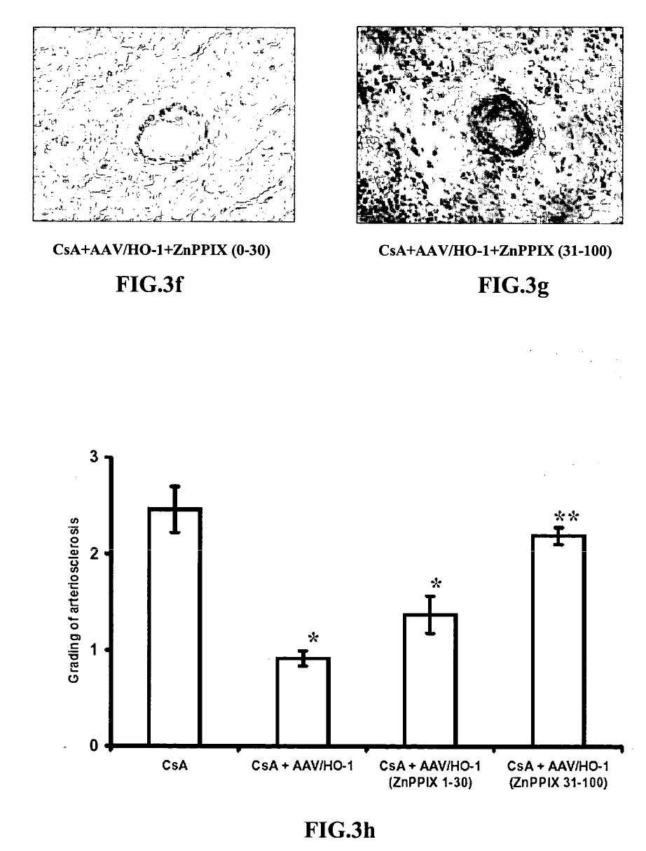

There are a number of advantages associated with use of the rAAV vector. rAAV HO-1 mediated expression exists in both endothelial cells of the graft vessels and cardiomyocytes in our model (FIG. 1). The stable persistence of carboxyhemoglobin in the peripheral blood of recipients (demonstrating the production of CO) demonstrate the stable increased activity of HO-1 in the grafts (FIG. 3). However, the ability to block the activity of HO allows us to assess when it is necessary to express HO-1 gene for the prevention of acute rejection and/or chronic rejection. If HO-1 gene activity is blocked in the early post-transplant phase, the survival of rAAV/HO-1 treated grafts is significantly reduced, suggesting that increased activity of HO in early post-transplant phase (day 0 to 30 in the model) is critical to protect grafts from acute rejection episodes. HO-1 expression leads to a remarkable reduction of infiltrating macrophages and T cells in the grafts and significantly brings down the production of anti-donor antibodies in the serum of the recipients. Although the mechanism of this effect has not been fully understood, the results indicate that HO-1 expression could modulate both the cellular and body-fluid immune responses that contribute to graft rejection. The extent to which HO-1 or its metabolic products (carbon monoxide, biliverdin/bilirubin and iron ions) contribute to modulate the immune response via suppression of endothelial cell activation and down-regulation of pro-inflammatory cytokines and chemokines still requires further investigation. Suggested in these findings is the possibility to modulate immune responses in recipients by expression of protective (anti-inflammatory) genes. HO-1 and its metabolic products, carbon monoxide and biliverdin/bilirubin, may exert anti-apoptotic and anti-inflammatory activities via the activation of the mitogen activated protein kinase (MAPK) p38 pathway, and the inhibition of macrophage to produce pro-inflammatory cytokines. HO-1 and carbon monoxide are also mediators of the anti-inflammatory effects of interleukin 10.

In the present study, direct evidence indicates that increasing the activity of HO-1 throughout the experiment, namely 100 days after transplant, is essential for the function improvement of grafts. The early expression of HO-1 (e.g., day 31-100 in the model) is apparently critical for survival of grafts, but less important for preventing the development of arteriosclerosis. In the three grafts that survive long-term despite early inhibition of HO-1 activity (e.g., in the first 30 days), the extent and obviousness of arteriosclerosis is no worse than arteriosclerosis in animals in which HO-1 is expressed for the whole post-transplant period of 100 days. It thus appears that an early increase of HO-1 activity after transplant is not essential for the prevention of arteriosclerosis in this model, while expression during the last 70 days suffices to suppress development of arteriosclerosis. This issue has been confirmed for all parameters measured including intimal proliferation of graft vessels and interstitial fibrosis (i.e., the consequence of tissue injuries and chronic inflammatory response).

In addition, increased activity of HO in the later post-transplant phase (e.g., day 31-100 after transplant) is as important as that in the early post-transplant phase for the prevention of grafts from intimal proliferation in vessels and interstitial fibrosis. The experiment also demonstrates that the transgene expression of HO-1 in endothelial cells can suppress intimal proliferation and fibrosis of vessels through up-regulation of p21.

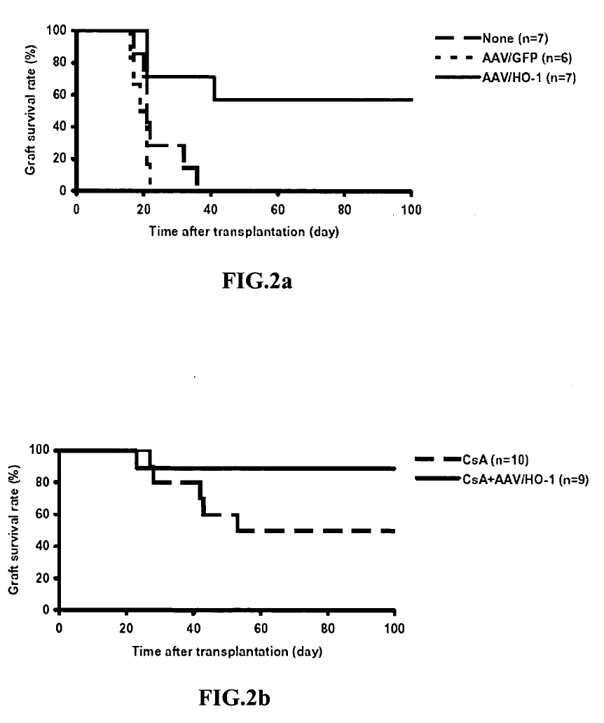

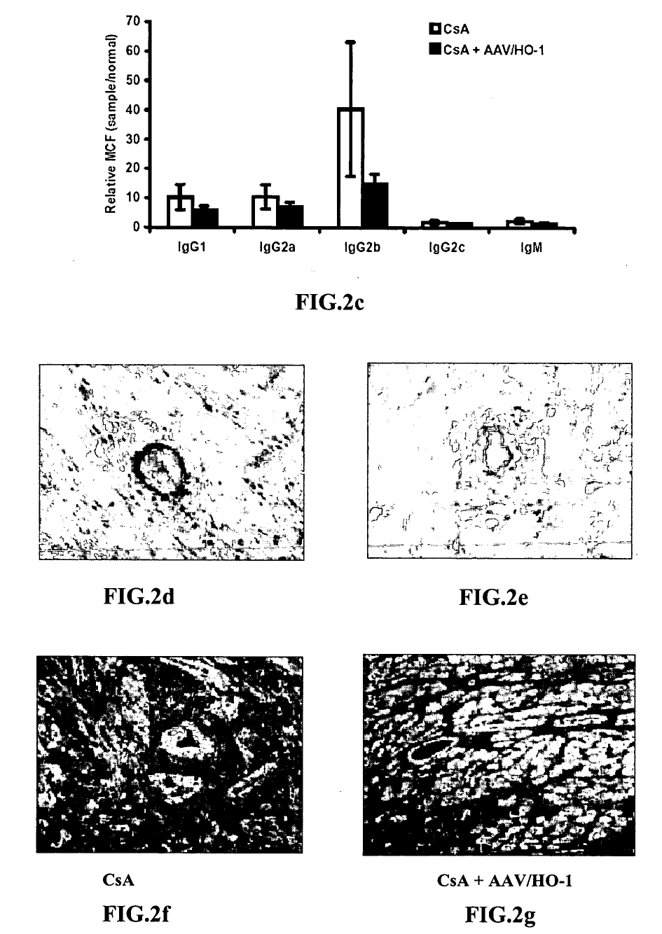

Here it is shown that rAAV/HO-1 gene transfer during cold organ preservation can alleviate graft arteriosclerosis and reduce the deposition of alpha-smooth muscle cell actin in graft vessels, thereby effectively preventing the development of intimal proliferation (FIG. 2). Although the precise mechanisms are still unclear, the up-regulation of p53 in all rAAV/HO-1 treated grafts and of p21 in some of the grafts displayed by western blot analysis (data not shown) might be functionally important for the inhibition of proliferating cells leading to vessel narrowing. It has been shown by recent studies that suppression of intimal proliferation involves up-regulation of p21. In addition, the increase in the numbers of apoptotic smooth muscle cells mediated by expression of HO-1 could also contribute to the inhibition of intimal proliferation.

Further, the study demonstrates that over-expression of TGF-β1 in grafts is associated with chronic organ dysfunction, which will lead to the deposition of fibrotic elements in the extracellular matrix. Down-regulation of these growth factors in rAAV/HO-1 treated grafts may, on the one hand, be a consequence of the anti-inflammatory effects of HO-1 which leads to a decrease in the number of infiltrating macrophages and thus a decrease in the production of TGF-β1. On the other hand, the study shows that the presence of HO-1 can down-regulate not only the intragraft expression of TGF-β1, but can also affect the signal transduction pathway of TGF-β1. HO-1 may down-regulate the expression of TGF-β1 by decreasing mRNA level of Smad 2 and Smad 3. Accordingly, HO-1 might down-regulate the expression of Smad-dependent PAI-1 via attenuation of nuclear translocation of Smad 2/4 proteins. This result is in accordance with a very recent study showing that CO can rescue the ischemia/reperfusion injury of lung via a mechanism associated with the down-regulation of PAI-1 in HO-1 deficient mice. The dynamic changes of gene expression related to extracellular matrix formation in the rAAV/HO-1 treated grafts might suggest that HO-1 can repress the undesirable effects of TGF-β1 on the deposition of extracellular matrix components.

In conclusion, long-term stable expression of HO-1 can prevent both acute and chronic allograft rejection in the model. In both early and late post-transplant phases, rAAV mediated over-expression of the HO-1 gene in grafts is critical for graft survival as well as improvement of transplant arteriosclerosis and interstitial fibrosis. The rAAV mediated transgene expression during organ preservation therefore represents a novel therapeutic approach to protect grafts from immune response and chronic tissue remodeling.

The specific procedures and methods supporting the study are as follows:

Rat HO-1 Gene Cloning and Construction of Vector Plasmid pSNAV1/HO-1

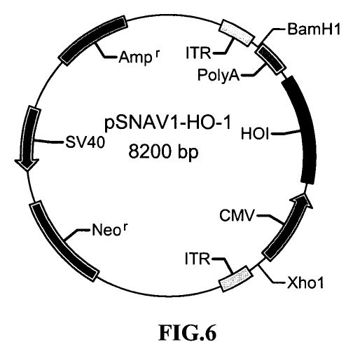

The rat HO-1 gene was cloned from a RT-PCR product of the rat spleen using pGEM-T plasmid (Promega). The key vector pSNAV1 was constructed from plasmids pSV2neo and pAV53. Vector pSNAV1 was formed by constructing in plasmid pAV53 the gene expression frame manipulated by human CMW promotor. Green fluorescent protein (GFP) or rat HO-1 gene was inserted into the multiple cloning site of pSNAV1 to construct pSNAV1/GFP or pSNAV1/HO-1. This is shown in FIG. 6.

Production of rAAV/HO-1 Viral Vector

The rAAV/HO-1 viral vector was manufactured using a method invented by WU, XiaoBing et al. (China Patent Appl. No.99119039.4, Publication No. 1252441A; China Patent Appl. No.99123723.4, Publication No. CN1272538A). Recombinant plasmid vectors pSNAV1/HO-1 were transferred into BHK-21 cells (purchased from ATCC) by using liposomes (lipofectamine) and resistant clones obtained by Neomycin selection. The cells transfected with recombinant plasmid vector pSNAV1/HO-1 can be selected for bearing the neo^{r} gene, which can be used to produce rAAV/HO-1 and designated as BHK/HO-1. After culture and subculture of the productive cells to set up cell banks, a large number of cells were obtained. rAAVIHO-1 can be rescued by using recombinant herpes simplex virus rHSV1-rc (described in China Patent of Appl. No.98120033.8, Pub. No. CN1248627A). rHSV1-rc contains the rAAV2-rep gene and the rAAV2-cap gene. rHSV1-rc is used to infect the above cells resulting in the production of a large amount of rAAV carrying the HO-1 gene and the gene expression frame thereof, namely rAAV/HO-1. rAAV/HO-1 was separated and purified according to the novel method for the purification of rAAV invented by Wu, Xiaobin et. al (China Patent Appl. No. 99123723.4). The titers of rAAV/GFP and rAAV/HO-1 vector (anti-DNase I granule) were detected with a dot hybridization technique by using digoxin-labeled CMV (cytomegalovirus) fragment probes. The infective titer of rAAV/GFP was detected by flow cytometric analysis.

Animals and Surgical Procedures

LEW (RT1′) and F344 (RT1^{lvl}) rats were purchased from the Institute of Laboratory Animals, Medical School Hannover, Germany, and maintained in the Laboratory Animal Unit, The University of Hong Kong. All experimental procedures were performed according to institutional guidelines and approved by the Committee on the Use of Live Animal in Teaching and Research. Allogeneic heart transplants were performed using LEW and F344 rats weighing from 200-250 grams as donors or recipients respectively. For the syngeneic graft, LEW rats were used as recipients. In brief, after in situ perfusion of donor heart grafts with the HTK solution (Koehler Chemie, Germany), 1×10¹² vector genomes of rAAV/GFP or rAAV/HO-1 were administered into the heart graft via the coronary artery. Grafts were then preserved in cold HTK solution for 6 hours before the transplant. The implantation procedure was as described in the literature. Graft function was monitored by daily palpation, and the allograft rejection was defined by full cessation of ventricular contraction, and confirmed by pathological examination.

Chemical Reagents

Cyclosporin A (CsA, Novartis Pharma., Switzerland) was given intramuscularly at a dose of 2.5 mg/kg/day to the animals on day 0-4 after transplantation. Heme oxygenase-1 inhibitor, zinc protoprophyrin IX (ZnPPIX, Calbiochem-Novabiochem, Germany) was administered intraperitoneally according to the experimental protocols.

Experimental Protocols

The experimental groups were as follows: 1) control, no treatment; 2) vector control, rAAV/GFP only; 3)rAAV/HO-1; 4) CsA, 2.5 mg/kg/d. i.m. day 0-4; 5)rAAV/HO-1+CsA, 2.5 mg/kg/d. i.m. day 0-4; 6)rAAV/HO-1+CsA (2.5 mg/kg/d. i.m. day 0-4)+ZnPPIX (2 mg/kg/d. day 0-30); 7)rAAV/HO-1+CsA (2.5 mg/kg/d. i.m. day 0-4)+ZnPPIX (2 mg/kg/d. i.p. day 31-60).

Histology, Immunohistochemistry and Western Blot Analysis

Long-term surviving grafts were removed on day 100, snap frozen and stored at −70° C. Haematoxylin & eosin staining, Verhoeff's haematoxylin staining or Masson trichrome staining were used to evaluate the histology of the grafts, deposition of fibrotic elements and elastin of the graft vessel. The grading of intimal proliferation of median arteries of the grafts after Verhoeff's haematoxylin staining was as follows: grade 0 (none), grade 0.5 (slight), grade 1 (5-25%), grade 2 (25-50%), grade 3 (>50%). The area of fibrotic tissue in the cross section of grafts was measured by computer software (Meta Morph imaging system, Universal Imaging Corporation, PA) after Masson's trichrome staining. All measurements were done in double blind manner with at least 50 arteries/graft (grading of intimal proliferation) and 20 areas/grafts (fibrotic area) in the continuing sections of 3-5 grafts/group. The phenotype of graft infiltrating cells was detected by immunohistochemistry using horseradish peroxidase protocol. Mouse anti-rat CD3 (G4.18), macrophage (ED1) and HO-1 (OSA-111) monoclonal antibodies (BD Pharmingen, Serotec and Stressgene) were used to detect graft infiltrating cells and HO-1 expression. The expression level of the HO-1 protein was determined by standard western blot analysis.

Measurement of HO Enzymatic Activity

The HO enzyme activity was measured by two of its end products: carbon monoxide and biliverdin/bilirubin. The amount of carbon monoxide was measured by spectrophotometry providing a determination of carboxyhemoglobin in the peripheral blood of the animals. The level of intragraft bilirubin was measured by the method as described. In brief, the frozen samples of grafts were homogenized in ice-cold sucrose and Tri-HCl buffer. The microsomal pellet was obtained after centrifugation, and re-suspended in MgCl₂-potassium phosphate buffer. Sample protein was then incubated with the reaction mixture containing rat liver cytosol, hemin, glucose-6-phosphate, glucose-6-phosphate dehydrogenase and NADPH (Sigma-Aldrich, St. Louis) for 60 minutes at 37° C. The generated bilirubin was measured by reacting with diazotized surfanilic acid to yield azobilirubin using spectrophotometry. The level of intragraft bilirubin was deduced by the ratio of its contents in the sample/naive heart.

Anti-Donor Antibodies

The serum of animals collected on day 100 were tested for various types of allo-specific antibodies by flow cytometric analysis. The cell suspension of the donor-type thymocytes was prepared first and incubated with heat-inactivated serum sample of recipients. Biotin-labeled monoclonal antibodies (BD Pharmingen) of anti-rat IgG1, IgG2a, IgG2b, IgG2c, IgM were used to detected the presence of various isotypes of alloantigen-specific antibodies. The level of each antibody was determined by the mean channel fluorescence and was calculated from ratio of sample/normal rat serum.

Profiling the Gene Expression

The gene expression profiles of grafts with and without rAAV/HO-1 treatment were determined by cDNA microarray. In brief, high purity of total RNA (Qiagen, Valencia) was isolated from whole grafts, reverse transcripted to cDNA and labeled with Cy3 or Cy5. Labeled probes were premixed and hybridized with the DNA sequences on the glass slides (Clontech, Palo Alto). Slides were scanned and the data were analyzed using Quantarray® software. All of the data was normalized by comparing with the signals of 9 housekeeping genes on the slides. A RNase protection assay was performed according to the protocol provided by manufacturer (RiboQuant kit, BD Pharmingen), briefly described as follows: 20 ug of RNA/sample was hybridized with complimentary [³²P]UTP labeled riboprobes, after the digestion with RNase, the protected probes were loaded on a denatured polyacrylamide gel and can be detected by exposure to X-ray film. Semi-quantitative RT-PCR was performed to detect the expression profiles of PAI-1, PAI-2, uPA and tPA in grafts on day 100.

Statistical Analysis

The survival rate data was evaluated by the Mann-Whitney U test, and the remaining data was analyzed using the Student's t-test. P<0.05 was considered statistically significant.

The following examples with reference to attached drawings describe the present invention in detail, and are not intended to limit the scope of the invention.

### Example 1

Stable Expression of Ho-1 Mediated by Adeno-Associated Viral Vector

Replication-deficient adeno-associated viral vectors were constructed by replacement of the genome of adeno-associated virus type 2 (except for 145-basepair ITR) with a gene expression cassette (FIG. 1a). The expression of rat HO-1 or green fluorescence protein (GFP) was under control of the human CMV promotor. With a rat heart transplantation model, the pattern of transgene expression and activity in syngeneic grafts following gene delivery (1×10¹² vector genomes) was investigated through cornary arteries during the cold presevation of the grafts. Expression of GFP was detected in the perivascular area on post-transplant day 7 (FIG. 1b). Immunohistochemical analysis demontrated that expression of HO-1 could also be detected in the endothelial cells of vessles of the grafts on day 7 (FIG. 1c). The expression of transduced GFP and HO-1, however, could only be detected in cardiomyocytes after 30 days (FIGS. 1d and e). Western blot analysis showed a consistent increase of HO-1 protein in the grafts (FIG. 1J). Next, HO-1 enzyme activity was determined based on two of its end-products: carbon monoxide and biliverdin (which can be quickly reduced to bilirubin by biliverdin reductase). The production of carbon monoxide was evaluated by measurement of carboxyhemoglobin in peripheral blood, showing a marked increase on day 14 (6.51±0.70%, n=5) and remained elevated at a stable level for over 3 months (FIG. 1g). Carboxyhemoglobin was undetectable in the GFP control group. The amount of bilirubin in the grafts on day 100 was also significantly increased in the rAAV/HO-1 treated heart grafts (1.32±0.09 fold-increase over a naive heart, n=5, p=0.0014) as compared with rAAV/GFP treated grafts (0.94±0.05, n=5) (FIG. 1h).

### Example 2

Induction of Long-Term Allograft Survival by Stable Expression of Ho-1

To determine the effects of rAAV mediated HO-1 gene expression in an allogeneic rat transplantation model, we transplanted LEW heart grafts to F344 recipients. In the absence of therapeutic intervention, LEW heart grafts were rejected with a median survival time (MST) of 21 days (n=7, FIG. 2a). Expression of rAAV/HO-1 in the heart graft prevented acute rejection and was associated with long-term survival (MST>100 days, n=7, P<0.05) in 4 of 7 animals, while in the control group of rAAV/GFP, all grafts were rejected in a similar pattern as the “no treatment” group (MST=20 days, n=6). Moreover, administration of a short course of CsA (2.5 mg/kg/day, day 0 to 4) to recipients significantly enhanced long-term graft survival of rAAV/HO-1 treated grafts, i.e. 8 of 9 grafts survived long-term (n=9), whereas merely 5 of 10 grafts survived long term after the same course of low-dose CsA (n=10, P<0.05, FIG. 2b).

To evaluate the long-term therapeutic effects of the rAAV/HO-1 treatment, various parameters in the grafts and the immune response of recipients were investigated on day 100. Impressively, the serum levels of anti-donor IgG1, IgG2b and IgM antibodies were remarkably reduced in rAAV/HO-1 treated animals (versus CsA treatment group, P<0.01, FIG. 2c). Moreover, there were fewer infiltrating mononuclear cells (T cells and macrophages) in the grafts of rAAVIHO-1 treatment group in comparison to that in the CsA treatment group on day 100 (data not shown). To determine the vascular lesion of grafts, the elastin in the arteries was stained on day 100 using Verhoeff's haematoxylin. In the CsA group, there was massive intimal proliferation and severe narrowing of the vessel lumen in median arteries of all grafts (grading of the vascular lesion=2.46±0.24, n=5, FIG. 2d), whereas no or minimal intimal proliferation was observed in the rAAV/HO-1 treated grafts (grading of vascular lesion=0.91±0.08, n=5, P<0.001, FIG. 2e). Meanwhile, the deposition of alpha-smooth muscle cell actin in the arterioles of grafts was also less in the rAAV/HO-1 treated grafts, indicating that the attenuation of intimal proliferation of the graft vessels by rAAV/HO-1 was achieved by the inhibition of proliferation of smooth muscle cells (FIGS. 2f and 2g).

### Example 3

The Role of HO-1 Expression on Graft Survival

To further investigate the function of long-term HO-1 expression mediated by rAAV in early and late post-transplant phases, we administrated zinc protoporphyrin IX (ZnPPIX, an HO inhibitor) intraperitoneally to the animals either in the early post-transplant phase (from day 0 to 30) or in the late post-transplant phase (from day 31 to 100). The efficiency of blocking HO activity was controlled by measuring the level of carboxyhemoglobin in the peripheral blood of recipients. Administration of ZnPPIX at a dose of 2 mg/kg/day to the recipients significantly inhibited the HO activity as reflected by a consistent low-level of carboxyhemoglobin equivalent to the control group. The activity of HO was restored after withdrawing ZnPPIX, appearing as the recovery of level of carboxyhemoglobin in the rAAV/HO-1 treatment group (FIG. 3a, b and c).

Afterwards, the importance of HO-1 expression at different post-transplant phases compared to graft survival was tested in the rAAV/HO-1+CsA group. Graft survival was shortened by combined administration of ZnPPIX from day 0 to 30 in the rAAV/HO-1 treated group (FIG. 3d). In contrast, graft survival rate remained unchanged when ZnPPIX was administered from day 31 to 100 (FIG. 3e). However, the increase of HO activity in the later post-transplant period significantly ameliorated transplant arteriosclerosis in terms of severity and number of diseased vessels (the grading of vascular lesion=1.37±0.19, n=3, P<0.001, FIG. 3j). Enhancement of HO-1 activity in early post-transplant period followed by blockade of it (day 31 to 100), however, failed to significantly improve the severity of arteriosclerosis (the grading of vascular lesion=2.19±0.09, n=3, P=0.07, FIG. 3g).

### Example 4

Reduction of Interstitial Fibrosis of Grafts in the Presence of HO-1 Expression

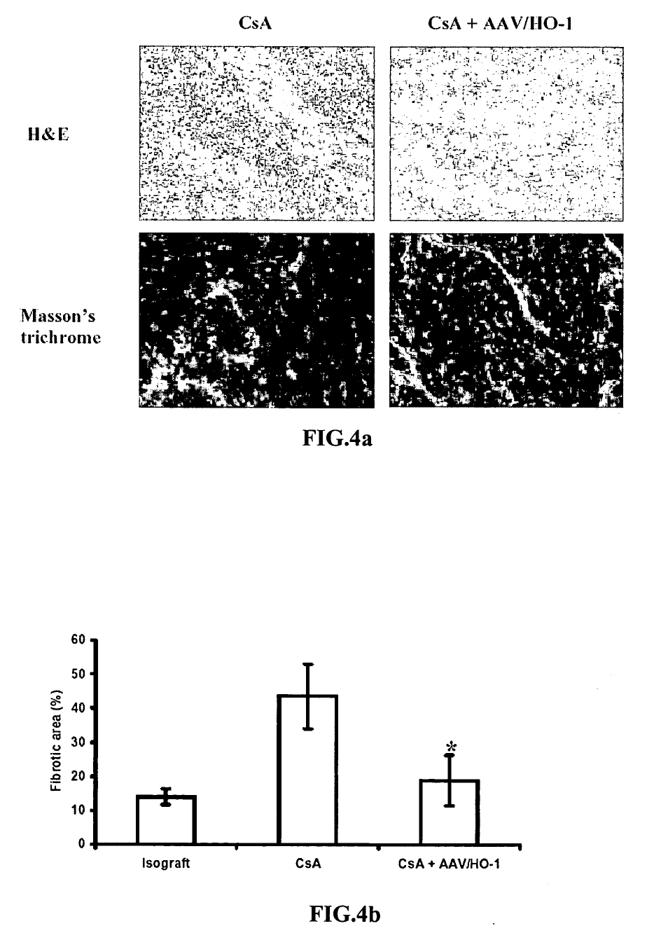

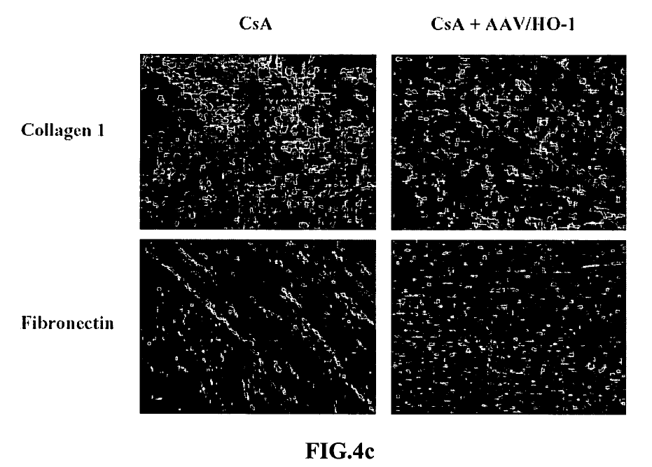

To determine the effect of long-term expression of HO-1 on the protection of cardiomyocytes from graft injury and interstitial fibrosis, the severity of fibrosis in grafts was evaluated by histology and immunocytochemistry. Massive fibrosis and necrotic cardiomyocytes were observed in the grafts of CsA treated animals on day 100, whereas the architecture of most cardiomyocytes was well preserved in rAAV/HO-1 treated grafts (FIG. 4a). Masson's trichrome staining showed significant reduction of fibrotic areas in rAAV/HO-1 treated grafts (19.0±7.44% versus 43.6±9.37%, n=5, P<0.01, FIG. 4b). Moreover, there was less deposition of fibronectin and a major component of fibrotic tissue, collagen I, in rAAV/HO-1 treated grafts on day 100 (FIG. 4c).

### Example 5

The Profiling of Gene Expression in Raav/Ho-1 Treated Grafts

To determine the effect of rAAV/HO-1 on gene expression, we analyzed the data of cDNA microarrays that compared the tissue samples of grafts with or without rAAV/HO-1 treatment under the same course of low-dose CsA. The data showed that rAAV mediated over-expression of HO-1 gene was associated with down-regulation of expressions of pro-inflammatory cytokines (e.g. tumor necrosis factor-alpha) and chemokines including macrophage inflammatory proteins (MIP) and macrophage migration inhibitory factor (MMIF)(fold _{log 2}<−1, Table 1). The expression level of transforming growth factor-beta 1 (TGF-β1), platelet-derived growth factor, fibroblast growth factor 2 and insulin-like growth factor were also lower in the rAAV/HO-1 treated grafts. In addition, mRNA transcriptions of interferon gamma, interleukin-2 and interleukin-2 receptor α chain were also under-expressed in the rAAV/HO-1 treated grafts, whereas interleukin-4 and interleukin-10 remained unchanged (data not shown). These findings were equally confirmed by either RNase protection assays or reverse transcript polymerase chain reactions (RT-PCR, data not shown).

### Example 6

Regulation of TGF-β Signaling Pathway

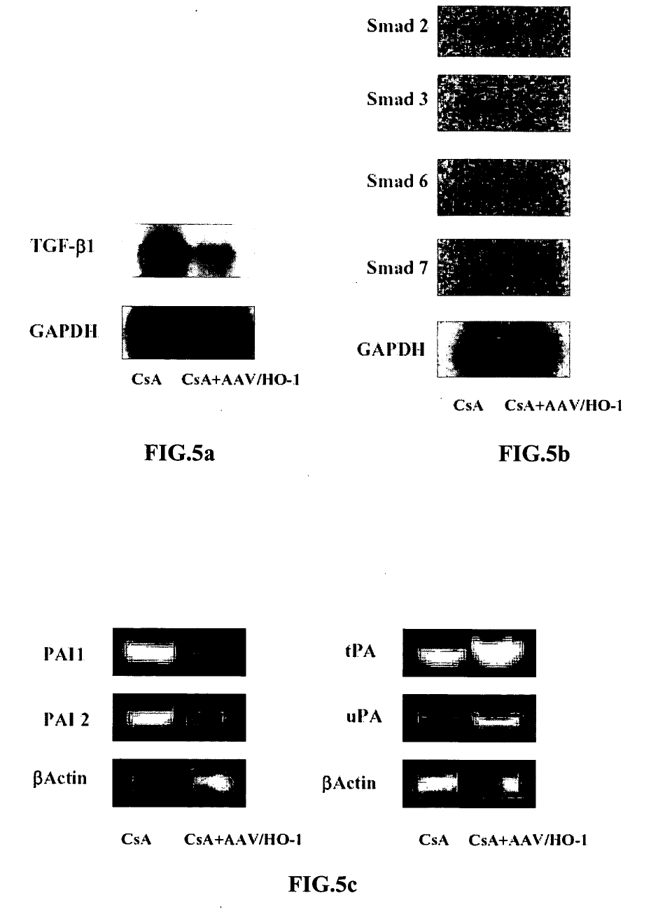

Excess TGF-β1 leads to disregulation of genes encoding proteins that regulate extracellular matrix formation. Long-term expression of HO-1 gene mediated by rAAV resulted in lower TGF-β1 mRNA expression in the grafts on day 100 (FIG. 5a). To further investigate the role of HO-1 in the regulation of TGF-β1 signal transduction pathway, the mRNA level of the members of the Smad protein family (e.g., human analogue of the mothers against decapentaplegic protein) was determined. Members of the Smad protein family are major mediators in the TGF-β1 signaling pathway. The results on day 100 showed that over-expression of HO-1 gene mediated by the rAAV was associated with remarkable under-expression of the intragraft mRNA level of Smad 2 and Smad 3, while Smad 6, a protein that inhibits the TGF-β1 signaling pathway, remained unchanged (FIG. 5b). In addition, both the cDNA microarray (Table 1) and the semi-quantitative RT-PCR showed under-expression of mRNA levels of plasminogen activator inhibitor-1 (PAI-1) and PAI-2 in the rAAV/HO-1 treated grafts on day 100 (FIG. 5c), whereas the mRNA levels of urokinase plasminogen activator (uPA) and tissue plasminogen activator (tPA) were over-expressed (FIG. 5d).

TABLE 1

| | | |
|---|---|---|
| | | Fold |
| Gene No. | Genes | (log 2) |

Proinflammatory cytokines and chemokines

| | | |
|---|---|---|
| R689 | Granulocyte-macrophage colony-stimulating factor | −1.91 |
| | (G-MCSF) | |
| RG434 | Macrophage inflammatory protein-2β (MIP-2β) | −1.79 |
| R534 | Macrophage inflammatory protein-3α (MIP-3α) | −1.62 |
| R579 | Tumor necrosis factor α (TNF-α) | −1.62 |
| R696 | Macrophage inflammatory protein-1α (MIP-1α) | −1.08 |
| R500 | Macrophage migration inhibitory factor (MMIF) | −1.05 |

Transforming growth factor superfamily

| | | |
|---|---|---|
| RG538 | Bone morphogenetic protein 4 (BMP-4) | −2.51 |
| RG243 | Bone morphogenetic protein 3 (BMP-3) | −2.16 |
| RG342 | Transforming growth factor β1 (TGF-β1) | −1.84 |
| R412 | Transforming growth factor β3 (TGF-β3) | −1.47 |
| RG539 | Bone morphogenetic protein 2 (BMP-2) | −1.18 |

Other growth factors

| | | |
|---|---|---|
| RG045 | Fibroblast growth factor 5 (FGF-5) | −1.76 |
| R128 | Fibroblast growth factor 10 (FGF-10) | −1.63 |
| R649 | Insulin-like growth factor 1B (I-LGF-1B) | −1.59 |
| R191 | Platelet-derived growth factor A (P-DGF-A) | −1.29 |
| R291 | Glioma-derived vascular endothelial cell | −1.26 |
| | growth factor (G-DVECGF) | |
| RG149 | Fibroblast growth factor 2 (FGF-2) | −1.23 |
| R251 | Insulin-like growth factor 2 (I-LGF-2) | −1.13 |

Protease inhibitors

| | | |
|---|---|---|
| RG358 | Calpastatin | −2.78 |
| RG526 | Plasminogen activator inhibitor 2A (PAI-2A) | −2.55 |
| R156 | Plasma protease inhibitor-α1 | −2.32 |
| R456 | Tissue inhibitor of metallproteinase 3 (TIMP-3) | −1.45 |
| R272 | Plasminogen activator inhibitor 1 (PAI-1) | −1.04 |

Serine proteases

| | | |
|---|---|---|
| R575 | Urokinase-type plasminogen activator (uPA) | 3.08 |
| RG058 | Dipeptidyl peptidase 4 | 2.30 |
| R271 | Tissue-type plasminogen activator (tPA) | 1.24 |
| R349 | Dipeptidyl peptidase 6 | 1.13 |

1. A recombinant plasmid vector pSNAV1/HO-1, comprising a heme oxygenase-1 (HO-1) gene.

2. An adeno-associated viral (AAV) vector cell strain, wherein the cell strain is obtained by transforming a cell with the recombinant plasmid vector of claim 1.

3. The AAV vector cell strain of claim 2, wherein the cell is a BHK-21 cell.

4. The AAV vector cell strain of claim 3, wherein the cell strain is BHK/HO-1.

5. A recombinant virus produced from the recombinant plasmid vector of claim 1.

6. A recombinant virus produced from the AAV vector cell strain of claim 2.

7. A recombinant virus produced from the AAV vector cell strain of claim 4.

8. The recombinant virus of claim 7, wherein the recombinant virus is rAAV/HO-1.

9. A process for the production of the recombinant adeno-associated virus rAAV/HO-1, the process comprising transforming a host cell with the recombinant plasmid vector pSNAV1/HO-1 having an HO-1 gene, and transfecting the host cell with recombinant virus HSV1-rc.

10. The process of claim 9, wherein the host cell is a BHK cell.

11. A method of mediating expression of the HO-1 gene, wherein the method comprises administering an effective amount of a recombinant adeno-associated viral vector.

12. A method of preventing post-transplant chronic transplant rejection, wherein the method comprises administering an effective amount of the recombinant virus of claim 5.

13. A method of preventing post-transplant chronic transplant rejection, the method comprising administering an effective amount of the recombinant virus of claim 6.

14. A method of preventing post-transplant chronic allograft rejection, the method comprising expressing the HO-1 gene in grafts.

15. The method of claim 14, wherein expression of the HO-1 gene in grafts is mediated by a recombinant adeno-associated virus.

16. The method of claim 14, further comprising constructing a plasmid bearing the HO-1 gene, and producing a recombinant adeno-associated virus bearing the HO-1 gene.

17. The method of claim 14, wherein expressing the HO-1 gene in grafts can be carried out by methods such as gene delivery method, protein delivery method and/or using substance for the induction of stable HO-1 expression.
